(19) United States
(12) Patent Application Publication
(10) Pub. No.: US 2017/0119885 A1
(43) Pub. Date: May 4, 2017

(54) OPHTHALMIC COMPOSITIONS AND METHODS FOR TREATING EYES

(71) Applicant: Hampton Technologies, Lynnfield, MA (US)

(72) Inventors: Edward J. Ellis, Lynnfield, MA (US); Jeanne Y. Ellis, Lynnfield, MA (US)

(21) Appl. No.: 15/306,329

(22) PCT Filed: Apr. 28, 2015
(86) PCT No.: PCT/US15/28015

§ 371 Date: Oct. 24, 2016

Related U.S. Application Data
(60) Provisional application No. 61/985,096, filed on Apr. 28, 2014.
(60) Provisional application No. 61/985,079, filed on Apr. 28, 2014.

(51) Int. Cl. A61K 47/18; A61K 47/38; A61K 47/36; A61K 9/00

(52) U.S. Cl. CPC A61K 47/186; A61K 9/0048; A61K 47/38; A61K 47/36

(57) ABSTRACT

Ophthalmic solutions are provided for use as tear substitutes and contact lens solutions. These solutions contain a cationic polymeric surfactant that has an affinity for the surface of the eye or a contact lens. Also disclosed are ophthalmic solutions containing a combination of the cationic polymeric surfactant and a water soluble anionic polymer which forms a complex in aqueous solution. The complex has an affinity for the surface of the eye or contact lens. In this manner, the present solutions provide improved duration of comfort to the user. The present compositions are also useful as carriers for ophthalmic drugs.

### CROSS-REFERENCE TO RELATED APPLICATION

The present invention claims priority to U.S. patent application Ser. No. 61/985,096, filed Apr. 28, 2014, and U.S. patent application Ser. No. 61/985,079, filed Apr. 28, 2014, each of which is hereby incorporated by reference in their entireties.

### BACKGROUND

Mammalian eyes, such as human and other mammalian (animal) eyes, advantageously are adequately lubricated to provide eye comfort and to more effectively provide good, clear vision. Ordinarily, such lubrication is obtained naturally from a tear film, which is formed over the outer, exposed ocular surface of the eye. This tear film is a complex fluid that is normally continuously replenished by the lacrimal, meibomian, and other glands, and when intact provides essential hydration and nutrients to the ocular surface. In addition to coating and protecting the delicate ocular surface, the tear film/air interface also serves as the initial refractive surface of the eye.

However, in many instances, the tear film is compromised by various etiological causes such as aging, hormonal deficiencies or changes, environmental factors (wind, heat, dust, cigarette smoke, hair dryers, etc.), a chronic low blink rate (VDT syndrome), contact lens wear, LASIK vision correction surgery, medications, and auto-immune diseases (lupus, rheumatoid arthritis and Sjogren's syndrome) (American Journal of Ophthalmology, 137, 337-342, 2004).

A relatively large number of compositions have been marketed for use in the treatment and management of the above mentioned conditions. These products are known by a number of names, for example; artificial tears, tear substitutes, lubricating drops, wetting drops and comfort drops. Many of these products have chemical compositions that attempt to emulate the composition and pH of normal tears and are intended to improve the continuous film and wetting over the corneal epithelial layer. For efficacy, the solutions often times may require instillation ten (10) times in a sixteen (16) hour day. Compositions which include specific lubricants have been utilized, for example, water soluble cellulosics, polyvinyl alcohol, polyvinyl pyrrolidone and polyethylene glycol. Although they wet the eye, their value in lubricating the eye is somewhat less than desired.

There have been recent attempts to provide improved ophthalmic solutions for treating various conditions of the eye including dry eye and computer vision syndrome. Some examples of the approaches taken are summarized below.

An ophthalmic composition disclosed in U.S. Pat. No. 7,875,271 comprises xanthan gum and glucose claiming to have a superior effect in treating corneal epithelial disorder. Moreover, this ophthalmic composition is in the form of an eye drop purported to have superior usability since it contains xanthan gum showing pseudoplasticity.

U.S. Pat. No. 8,211,942 teaches an approach that does not follow the reductionist paradigm, nor does it focus on a specific effect, such as adjusting tonicity, enhancing lubrication by augmenting and maintaining a stable tear film over the ocular surface, adding a positively or negatively charged complex of phospholipids to the ocular surface of the eye, maintaining mucin goblet cells, and the like. Instead, the subject matter provides compositions, e.g., pharmaceutical compositions, useful in a variety of applications, wherein the components of the composition are primarily non-ionic and suitable in compositions with other components, e.g., drugs or other bioactive molecules. In one embodiment, the disclosed compositions comprise a plurality of components, the majority of which are non-ionic in nature, which results in a particularly biocompatible composition.

U.S. Pat. No. 8,569,370 and 20140045939 disclose methods of treating dry eye, wherein the method comprises administering a topical ophthalmic composition comprising a therapeutically effective amount of each of: an aqueous carrier; erythritol, and its isomers thereof; carnitine, its isomers or suitable salts thereof; glycerin; and an ionically charged polymeric material selected from the group consisting of carboxymethyl cellulose and mixtures of carboxymethyl cellulose compounds.

U.S. Pat. No. 8,664,197 discloses ophthalmic solutions containing arabinogalactans with a protective activity on the corneal epithelium, particularly suitable for use as artificial tears stimulating the recovery of corneal lesions and also particularly useful for contact lens users. The compositions according to the invention have a virtually negligible viscosity, but are sufficiently mucoadhesive to assure a considerable permanence time in the area of application. Besides being well-tolerated, the aforesaid compositions have considerable re-epithelization capacity

U.S. Pat. No. 7,914,803: 7,947,295; 6,838,449; 6,583,124 and 6,403,609 are directed to ophthalmic compositions containing a gelling amount of a combination of galactomannan polysaccharides and borates. The compositions gel or partially gel upon administration to the eye. Commercial products resulting from these patents have been developed by Alcon Laboratories and are marketed as Systane® ocular lubricants.

U.S. Pat. Nos. 4,914,088; 5,278,151; 5,294,607; 5,371,108; 5,578,586 and 20090068237 disclose the use of lipids in the form of an emulsion composition, generally based on phospholipid technology, for the formation of an artificial tear film over the ocular surface of the eye capable of providing mechanical lubrication for the ocular surface while reducing evaporation of fluid there from. The emulsion is desirably in the form of a meta stable emulsion and is characterized by the use of a surfactant comprising a combination of a primary and secondary surfactant where the primary surfactant permits formation of the emulsion and the secondary surfactant permits autoclaving of the surfactant. A commercial ophthalmic solution purported to be the “first multi-dose, emollient-based lipid restorative tear” emerged with Alimera Sciences' launch of Soothe® in 2005 (now marketed by Bausch &Lomb as Soothe XP®). The foundation of this tear is its ability to bolster the lipid components of the tear film via the lipid emulsion formulation.

Application U.S. 20080050335 discloses aqueous ophthalmic solutions containing a combination of hyaluronic acid or a pharmaceutically acceptable salt thereof, e.g. sodium hyaluronate, and polyvinyl alcohol. These solutions are claimed to have a synergistic effect on viscosity and provide a statistically significant improvement over the prior art formulations. The compositions are said to be useful as artificial tear solutions for the treatment of dry eye syndrome and ocular discomfort and may be administered whenever the use of artificial tears is advisable.

U.S. Pat. No. 8,455,462 discloses ophthalmic compositions based on tamarind seed polysaccharide and hyaluronic acid. More particularly, the invention concerns ophthalmic solutions indicated for use as tear substitutes, containing a combination of hyaluronic acid and a polysaccharide known as TSP (Tamarindus indica Seed Polysaccharide) which are able, when administered together in a combination, to act synergistically in stimulating the return to normality in the conjunctival mucosa affected by the dry eye syndrome, thus inducing a remarkable improvement in the number and morphology of the conjunctival microvilli.

U.S. Pat. No. 8,524,779 discloses pharmaceutical, ophthalmic or cosmetic oil-in-water emulsion compositions containing quaternary ammonium compounds, more preferably to ophthalmic emulsions being useful for eye care or for the treatment of eye conditions. This patent also relates to compositions including at least one quaternary ammonium compound as cationic agent. The patent states that cationic emulsions have better spreading coefficients across the cornea and conjunctiva vs. conventional eye drops and anionic emulsions. Improved spreading coefficient leads to better ocular surface wettability, and electrostatic attraction reduces tear washout. In oil emulsions, the electro-attractive interactions between the positively charged oil droplets of the cationic emulsion and the negatively charged ocular surface effectively lower the contact angle and increases the dwell time of the drop. This technology has been commercialized by Novagali Pharma (Cationorm) marketed in the United States as Retaine MGD (Ocusoft)®—is the first cationic emulsion technology introduced to the market specifically for treatment of dry eye. Retaine MOD® is a preservative-free, hypotonic, oil-in-water emulsion based on a positively charged emulsion.

Presently artificial tears are the mainstay for providing comfort and relief to patients with a compromised ocular surface. In addition to being safe and effective, the ideal topical ocular lubricant is characterized by its ability to spread efficiently, quickly, and evenly over the cornea, to minimize friction between the upper eyelid and the cornea, to cause minimal blur upon instillation, and to improve both the subjective symptoms and the objective signs of a distressed ocular surface. While many attempts have been made to prolong the lubricating effects of an eye drop little progress has been achieved. Therefore, the need for a longer lasting ocular lubricant that provides the user comfort remains a challenge.

Hyaluronic acid (sodium hyaluronate) has emerged as a useful ingredient in cosmetic products especially skin creams and more recently ocular products. Hyaluronic acid or sodium hyaluronate is an innovative new visco-enhancer for use in topical eye care formulations. It is produced by fermenting the safe bacterial strain Bacillus subtilis—the world's first hyaluronic acid that is 100% free of animal-derived raw materials and organic-solvent remnants. For the purposes of this invention “hyaluronic acid” and “sodium hyaluronate” refer to the same polymer namely sodium hyaluronate.

Hyaluronic acid is a key comfort ingredient for topical ophthalmic formulations since it is a natural compound that is biocompatible, non-immunogenic, and biodegradable. In fact, it is one of the most hygroscopic molecules found in nature; hydrated hyaluronic acid can contain up to 1,000-fold more water than its own weight. These exceptional water retention properties result in enhanced hydration of the corneal surface. Moreover, applications of ophthalmic formulations containing hyaluronic acid reduce tear elimination and enhance precorneal tear film stability, which is a useful property against dry eye syndrome.

The non-Newtonian and shear-thinning properties hyaluronic acid produce solutions with a high viscosity at low shear rate (when the eye is open) and a low viscosity at high shear rate (during blinking), facilitate even distribution of the solution and lubrication of the ocular surface. Moreover, the muco-adhesivity of hyaluronic acid provides effective coating and long-lasting protection of the cornea as well as extended residence times on the ocular surface. Finally, when topically instilled on the eye, hyaluronic acid promotes physiological wound healing by stimulating corneal epithelial migration and proliferation of keratocytes as well as reducing the healing time of corneal epithelium.

The capacity of hyaluronic acid of various chain lengths to bind water was assessed from the amount of non-frozen water in Differential Scanning calorimetry (DSC). Overall, the results of our studies show that all hyaluronic acid samples bind very high amounts of water: 4-5 g/g. Moreover, the amount of bound water is independent of the molecular weight and the origin of the hyaluronic acid. When topically applied to the eye, hyaluronic acid enhances water retention on the corneal surface and increases corneal wettability thanks to its superior water retention properties.

The corneal residence time of hyaluronic acid at 0.1% (w/v) in topical ophthalmic solutions with a drug, hyaluronic acid can increase the contact time with the ocular surface, thereby improving the bioavailability of the drug.

The rheological properties of eye drops and artificial tears largely depend on the concentration, the molecular weight, and the nature of the viscosity-enhancing agents. As the viscosity-enhancing agent of choice, hyaluronic acid decreases the drainage rate of ophthalmic solutions. In eye drops designed for drug delivery, a highly viscous hyaluronic acid solution prolongs the contact time of the drug with the cornea, resulting in improved bioavailability of the drug. But there can also be disadvantages related to the use of thickened eye drops, including ocular discomfort, increased eye blink frequency, and possible interference with vision. Solutions containing hyaluronic acid feature well-defined rheological properties while allowing for maximum comfort and efficacy.

The positive attributes of hyaluronic acid based solutions are balanced by its lack of surface activity especially with somewhat hydrophobic surfaces such as the cornea and contact lenses. The result is that the comfort provided by hyaluronic acid is short lived since it is flushed from the eye quickly by the tear fluid turnover. It that respect hyaluronic acid is much like other anionic polymers that are utilized in ophthalmic solutions.

There are several hyaluronic acid based ophthalmic solutions on the market as lubricating drops and as contact lens solutions. These would include Blink Tears® Lubricating Eye Drops from Abbott Medical Optics Inc; Biotrue® multi-purpose solution from Bausch & Lomb; I-Drop® from I Med for dry eye and Aqualarm® UP from Bausch & Lomb. There has been a limited amount of work to promote the binding of hyaluronic acid to the surface of a contact lens. Patent application U.S. Appl. 20100178317, now under appeal, describes the use of an amphoteric surfactant as a means to promote binding of hyaluronic acid to the surface of a contact lens. The examples center around hydrogel lenses. Another patent, U.S. Pat. No. 6,277,365, describes the use of a cationic glycoside surfactant a means to promote binding of hyaluronic acid to the surface of a contact lens. The examples in this application center on RGP lenses.

A review of the patent literature dating back to the 1970's reveals an approach to improve the attraction of a polymer solution ingredient to the surface of the eye or a contact lens. In this manner the eye or lens surface would be made wettable and therefore comfortable. The approach was based on the fact that most surfaces found in nature are slightly anionic. Most contact lens surfaces also display this characteristic. The approach taken was to utilize a water soluble cationic polymer to bind to the surface of a contact lens or the eye itself. The polymer chosen was commercially available cationic cellulose. The theory was that this cationic polymer would bind to the anionic surface of a contact lens and provide wettability and longer term comfort to the lens wearer. The in practice the binding strength of the cationic polymer to the surface by electrostatic forces was found to be weak. This coupled with the fact that ophthalmic solutions contain salts and buffers tended to screen the cationic polymer from the surface. For reference purposes the following U.S. Pat. Nos. 4,321,261; 4,436,730; 5,401,327; 5,500,144; 5,604,189; 5,711,823; 5,773,396; 5,872,086; 6,096,138; 6,348,508; 8,664,180; 20020115578; 20050119221; 20050202986 and 20130202547.

The compositions of this invention, in the form of a solution, are useful to provide long lasting comfort to dry eye sufferers, computer users and also are useful as contact lens solutions for rigid gas permeable lenses and soft lenses including silicone hydrogels.

### SUMMARY

New ophthalmic compositions for treating eyes and methods of treating eyes have been discovered. The present invention relates to ophthalmic compositions and methods useful for treating eyes. More particularly, the present invention relates to ophthalmic compositions including mixtures of components which are effective in providing desired protection to ocular surfaces of human or animal eyes, and to methods for treating human or animal eyes using the present ophthalmic compositions. In another aspect the disclosed ophthalmic solutions are also useful as a carrier of ophthalmic drugs.

The present compositions, in solution form, very effectively treat eyes, for example, eyes afflicted or susceptible to diseases/conditions, such as, without limitation, dry eye syndrome, low humidity environments, and stress/trauma, for example, due to surgical procedures, and the like. In particular, these solutions would be useful for mitigating the damaging effects of environmental factors (wind, heat, dust, cigarette smoke, etc.) and chronic low blink rate (CVS syndrome). The ophthalmic solutions of this invention are particularly useful as contact lens solution for both rigid and soft lenses. The present solutions are relatively straightforward, can be easily and cost effectively manufactured, and can be administered, for example, topically administered, to an ocular surface of an eye very conveniently. The present solutions are useful as a carrier for delivering drugs, particularly hydrophobic drugs, to the ocular surface.

In one broad embodiment of the present invention ophthalmic compositions are provided comprising a carrier component, advantageously an aqueous carrier component and a cationic polymeric surfactant.

In a second broad embodiment of the present invention ophthalmic compositions are provided comprising a carrier component, advantageously an aqueous carrier component a cationic polymeric surfactant and a water soluble anionic polymer which forms a surface active complex with the cationic polymeric surfactant.

This invention describes ophthalmic solutions comprising a carrier, for example, an aqueous carrier component, and an effective amount of a material selected from among, but not limited to:
Buffering agents
Tonicity adjusting agents
Viscosifiers (demulcents)
Chelating agents
Anti-microbial agents/Preservatives/Chelating agents
Vitamins/Minerals
Cooling agents
Radiation absorbers
Stabilizers/Anti-oxidants
Immunosuppressive agents

In yet another broad aspect of the present invention, ophthalmic solutions are provided comprising a carrier component, advantageously an aqueous carrier component, a cationic polymeric surfactant and an ophthalmic drug.

Methods of treating human or animal eyes are also provided. Such methods comprise administering a solution, for example, a solution in accordance with the present invention, to a human or animal eye to provide at least one benefit to the eye. Any and all features described herein and combinations of such features are included within the scope of the present invention provided that the features of any such combination are not mutually inconsistent.

Further provided is a method to provide comfort and relief to individuals with ocular irritation. The method includes contacting an ophthalmic surface with an effective amount of a solution of this invention administered in drop or mist form either from a multi-dose container or in a unit-dose container.

This Summary is an overview of some of the teachings of the present application and not intended to be an exclusive or exhaustive treatment of the present subject matter. Further details about the present subject matter are found in the detailed description and appended claims. Other aspects of the invention will be apparent to persons skilled in the art upon reading and understanding the following detailed description and viewing the examples that form a part thereof, each of which are not to be taken in a limiting sense. The scope of the present invention is defined by the appended claims and their equivalents.

### DETAILED DESCRIPTION OF CERTAIN EMBODIMENTS

Unless defined otherwise, all technical and scientific terms used herein have the same meaning as commonly understood by one of ordinary skill in the art to which this invention belongs. Generally, the nomenclature used herein and the laboratory procedures are well known and commonly employed in the art. Conventional methods are used for these procedures, such as those provided in the art and various general references. Where a term is provided in the singular, the inventors also contemplate the plural of that term. The nomenclature used herein and the laboratory procedures described below are those well known and commonly employed in the art.

The present invention is directed to ophthalmic compositions, ophthalmic solutions, gels or ointments or dispersions which, in one embodiment, comprise a cationic polymeric surfactant and in a second embodiment comprise a complex formed of a cationic polymer surfactant and an anionic polymer. The present invention is also directed to methods of using these compositions to treat various ophthalmic disorders including dry eye, glaucoma, ocular hypertension, infection, allergy, inflammation and VDT syndrome. The ophthalmic compositions of this invention are also useful as the basis for contact lens solutions.

With respect to the first embodiment the ophthalmic compositions of this invention comprise a cationic polymeric surfactant. Given the tools available to the polymer chemist and the wealth of available monomers and natural compounds an endless number of cationic polymers can be synthesized. For the purposes of this invention one class of polymers is of particular interest and that class is polysaccarides. The most important chemical building block of the polysaccharides (e.g. starch, cellulose, and guar) is the anhydroglucose unit (AGU). See structure below:

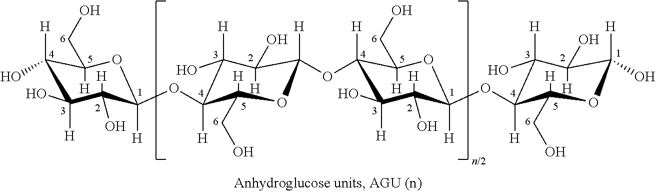

These units contain multiple hydroxyl groups that can be derivatized to yield polymers with a wide variety of physical and chemical properties. One important class of such polymers is the cationic polysaccharides which include hydrophobic cationic polysaccharides. Cationic cellulose and hydrophobic cationic cellulose are of particular interest in the practice of this invention. See “FUNCTIONALIZATION OF CELLULOSE: SYNTHESIS OF WATER-SOLUBLE CATIONIC CELLULOSE DERIVATIVES”; Jolanta Liesiene and Jurgita Kazlauske: CELLULOSE CHEMISTRY AND TECHNOLOGY: 47 (7-8) 515-525 (2013)(which is hereby incorporated by reference in it entirety).

There are a number of commercially available cationic polymers, including cellulose, available. The more common ones have been categorized “Polyquaternium” which is the International Nomenclature for Cosmetic Ingredients designation for many cationic polymers that are used in the personal care industry. Polyquaternium is a neologism used to emphasize the presence of quaternary ammonium centers in the polymer. INCI has approved a number of different polymers under the Polyquaternium designation. Different polymers are distinguished by the numerical value that follows the word “Polyquaternium”. Polyquaternium-5, polyquaternium-7, and polyquaternium-47 are three examples, each a chemically different type of polymer. The numbers are assigned in the order in which they are registered rather than because of their chemical structure.

Polyquaterniums find particular application in conditioners, shampoo, hair mousse, hair spray, hair dye, and contact lens solutions. Because they are positively charged, they neutralize the negative charges of most shampoos and hair proteins and help hair lie flat. Their positive charges also ionically bond them to hair and skin.

While there are many positively charged water soluble polymers there are far fewer positively charged polymeric surfactants. From a commercial standpoint there are three notable examples of cellulose based cationic polymeric surfactants. For a detailed description of cationic cellulose surfactants see U.S. Pat. No. 4,663,159 and U.S. Pat. No. 7,868,164. These patents are hereby incorporated by reference in their entirety. It should be understood that the following examples are not meant to limit the scope of this invention. The first example is Polyquaternium 24, Cas Number 98616-25-2. The chemical name is: Cellulose ether with .alpha.-(3-(dodecyldimethylammonio)-2-hydroxypropyl)-.omega.-hydroxypoly(oxy-1, 2-ethanediyl) chloride. The structural formula is:

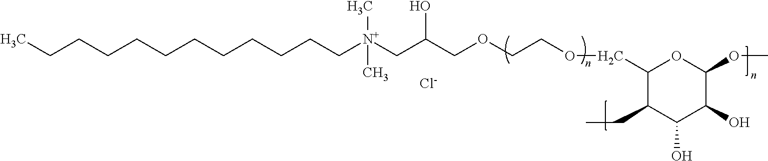

Polyquaternium 24 has been sold by Amerchol under the trade name Quatrisoft LM-200®. A newer line of cationic polymeric surfactants is now offered by Amerchol under the trade name SoftCAT®. These polymers have been classified by INCI as Polyquaternium 67. The chemical name is: 2-Hydroxyethyl cellulose ether, reaction products with N,N,N-trimethyl-N-oxiranylmethylammonium chloride and N-dodecyl-N,N-dimethyl-N-oxiranylmethylammonium chloride.

Another source of cationic polymeric surfactants is Croda Inc. under the trade name of Crodacel®. There are three products available:
Crodacel QS—PG-hydroxyethylcellulose stearyldimonium chloride
Crodacel QM—PG-hydroxyethylcellulose lauryldimonium chloride
Crodacel QL—Lauryldimonium hydroxypropyloxyethyl cellulose.

However, it should be noted that the invention is not limited to these polymers.

The above mentioned cationic polymeric surfactants are particularly useful in the practice of this invention. These surfactants provide a means binding the polymer to the surface of the eye to achieve improved wettable and thus increase comfort to the patient. The same principle applies to contact lenses, especially rigid gas permeable lenses, where the polymeric surfactant binds to the lens surface thus providing improved wettability and lens comfort.

The first embodiment of this invention describes the incorporation of a cationic polymeric surfactant into an aqueous ophthalmic composition. When utilized, the ophthalmic composition will facilitate the adsorption of the cationic polymeric surfactant onto the eye surface or onto a contact lens surface to achieve longer lasting wettability and comfort for the patient.

Preferably, the cationic polymeric surfactant is a hydrophobe substituted, cationic polysaccharide and comprises from about 0.01 to about 5.0 weight percent of the ophthalmic composition. Preferably, the cationic polymeric surfactant is a hydrophobe substituted, cationic cellulose and comprises from about 0.01 to about 5.0 weight percent of the ophthalmic composition. Preferably, the cationic polymeric surfactant is a hydrophobe substituted, cationic cellulose and comprises from about 0.05 to about 5.0 weight percent of the ophthalmic composition. More preferably, the hydrophobe substituted, cationic cellulose comprises from about 0.05 to about 3.0 weight percent of the ophthalmic composition. Most preferably, hydrophobe substituted, cationic cellulose is Polyquaternium 24 or Polyquaternium 67 and comprises from about 0.05 to about 3.0 weight percent of the ophthalmic composition.

With respect to the second embodiment the ophthalmic compositions of this invention comprise a cationic polymeric surfactant complexed with an anionic water soluble polymer. This type of structure is often referred to as a polyelectrolyte complex. This invention utilizes an anionic polymer to form a unique surface active complex with the cationic polymeric surfactant to provide a protective coating on the ocular surface or on a contact lens surface. The anionic polymer must be readily water soluble and present a proper charge structure to allow the complex to remain soluble in an aqueous environment. While there is a wealth of anionic polymers available certain commercially available polymers are particularly useful in the practice of this invention. Examples of such anionic polymers are:

Carboxymethyl cellulose
Carboxymethylhydroxyethyl cellulose
Carboxyethylhydroxyethyl cellulose
Carboxymethyl guar
Carboxymethylhydroxypropyl guar
Cellulose phosphate
N-Carboxymethyl chitosan
Alginates, sodium or potassium salts
Xanthan, sodium or potassium salts
Hyaluronic acid, sodium or potassium salts

### Glycomacropeptide

Other anionic polysaccharides

Listed above is gylcomacropeptide an anionic peptide. Bovine glycomacropeptide (GMP) is the hydrophilic C-terminal peptide produced from κ-casein during cheese-making. N-Acetylneuraminic acid (NeuNAc) is one of the sugars associated with bovine GMP and has been reported to contribute both biological and functional properties to this dairy peptide.

Glycomacropeptide (GMP) has many unique characteristics when compared to other whey proteins. The “glyco” portion of glycomacropeptide refers to the saccharide groups that are attached to the peptide backbone of the molecule. Glycomacropeptide contains various amounts of covalently attached oligosaccharides, including N-acetylneuraminic acid (sialic acids), galactose and N-acetyl-galactosamine. The most prominent of theses is sialic acid, which comprises about 7-8% of the glycomacropeptide.

The above mentioned anionic polymers are useful in the practice of this invention. However, it should be noted that the invention is not limited to these polymers.

Hyaluronic acid (sodium hyaluronate) is a particularly useful polymer in the practice of this invention. Hyaluronic acid is a non-immunogenic substance and because of its viscoelastic and hydrophilic properties hyaluronic acid has been used for many years as an eye vitreous or joint fluid replacement or as a supportive medium in ophthalmic surgery. The use of hyaluronic acid in ophthalmic solutions provides a very comfortable and smooth feeling when instilled in the eye. These solutions have been widely used as artificial tear drops for treating patients with dry eye. The benefits derive, in part, from the water binding capacity of hyaluronic acid. Studies have demonstrated that hyaluronic acid binds a large amount of water that is not dependant on molecular weight. The water retentive properties of hyaluronic acid enhances and sustains moisturization of the ocular surface and contributes to the stabilization of the of the precorneal tear film. Unfortunately, the beneficial attributes of hyaluronic acid eye drops are short lived. This is due to the fact that hyaluronic acid is not surface active and will not adhere to the cornea. According to studies the turnover of tears in the eye is about 15 minutes. Most artificial eye drops, including hyaluronic based drops, will only provide very temporary duration of action.

In the case of contact lens solutions based on hyaluronic acid the same lack of surface activity prevents the hyaluronic acid from binding to the surface of a contact lens. When the lens is placed in the eye the hyaluronic acid dissipates just as quickly as eye drops.

Clearly, there is a need to prolong the beneficial effects of hyaluronic acid by providing a means to improve the residence time in the eye or on a contact lens surface. This patent application teaches the use of a polymer complex to substantially improve binding of hyaluronic acid to the surface of the eye and to contact lenses.

Hyaluronic acid is an anionic, nonsulfated glycosaminoglycan having the following general structure:

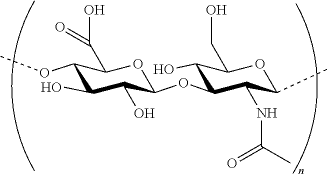

Hyaluronic acid is a polymer that has a very wide molecular weight range spanning from about 50×10⁴ daltons to about 2-4×10⁶ daltons. The higher molecular weight hyaluronic acid provides unique rheological properties. In the practice of this invention the hyaluronic acid utilized to produce the complex preferably has a molecular weight of from about 200,000 to about 4,000,000 daltons. Preferably, the range is from about 750,000 to about 2,000,000 daltons. More preferably, the range is from about 800,000 to about 1,750,000 daltons. An even more preferred range is from about 900,000 to about 1,500,000 daltons.

The second embodiment of this invention describes the incorporation of an anionic polymer to complex with a cationic polymeric surfactant in an ophthalmic composition.

It is a feature of this invention that a complex between a cationic polymeric surfactant and hyaluronic acid can be obtained commercially and utilized in the practice of this invention. Biopolymer HA-24 Bio® is an association complex between Polyquaternium-24 and hyaluronic acid sold by Lipo Chemical. Amerchol offers MOCARE Polymer HA-24® which is an association complex between Polyquaternium-24 and hyaluronic acid. Dow Consumer & Industrial Solutions has recently introduced an association complex comprised of hyaluronic acid and polyquaternium-67 product name “MoistStar HA+® Moisturizing Technology”.

It has been reported that Biopolymer HA-24® has a composition of 50% by weight of hyaluronic acid (50% Polyquaternium-24) and MoistStar HA+® has a composition of 43% by weight of hyaluronic acid (57% Polyquaternium-67).

When utilized in an ophthalmic solution the polymeric complex will bind to the eye surface or to a contact lens surface to achieve longer lasting wettability and comfort for the patient.

Preferably, the polymeric complex will be formed from a weight ratio of about 1 to 10 to a ratio of about 10 to 1 of cationic polymeric surfactant to the anionic water soluble polymer. Preferably, the polymeric complex comprises from about 0.01 to about 5.0 weight percent of the ophthalmic solution. More preferably, the polymeric complex comprises from about 0.05 to about 3.0 weight percent of the ophthalmic solution. Most preferably, the cationic polymeric surfactant is Polyquaternium 24 or Polyquaternium 67 and the anionic polymer is hyaluronic acid (sodium hyaluronate) and said complex comprises from about 0.05 to about 3.0 weight percent of the ophthalmic solution.

With respect to the first and second embodiment of the present invention the ophthalmic compositions described herein contain other components to provide key attributes to the solution product. The following solution components may be utilized in the practice of this invention

Generally, ophthalmic compositions, including eye solutions, contain a buffer to adjust the pH of the product to ensure compatibility with the eye tissue. The usual pH range from about pH of 6.0 to about pH of 8.0.

Typical buffering systems include: borate buffers, for example, boric acid and its salts, for example, sodium borate or potassium borate. Borate buffers also include compounds such as potassium tetraborate or potassium metaborate that produce borate acid or its salt in solutions. Borate buffers are known for enhancing the efficacy of certain polymeric biguanides. For example, U.S. Pat. No. 4,758,595 to Ogunbiyi et al. describes that a contact-lens solution containing a polyaminopropyl biguanide (PAPB), also known as PHMB, can exhibit enhanced microbial efficacy if combined with a borate buffer.

A phosphate buffer system preferably includes one or more monobasic phosphates, dibasic phosphates and the like. Particularly useful phosphate buffers are those selected from phosphate salts of alkali and/or alkaline earth metals. Examples of suitable phosphate buffers include one or more of sodium dibasic phosphate, sodium monobasic phosphate and potassium monobasic phosphate.

Other known buffer compounds can optionally be added to the lens care compositions, for example, citrates, citric acid, sodium bicarbonate, TRIS, acetate and the like. Other ingredients in the solution, while having other functions, may also affect the buffer capacity. For example, EDTA, often used as a complexing agent, can have a noticeable effect on the buffer capacity of a solution.

Also useful are combination buffer systems, for example, boric acid/borate or a combined boric/phosphate buffer system. For example a combined boric/phosphate buffer system can be formulated from a mixture of sodium borate and phosphoric acid, or the combination of sodium borate and the monobasic phosphate. Another particular buffer system is based on diglycine. Diglycine can be used in the composition as the sole buffer system or in combination with another buffer system.

Buffering systems such as, but not limited to, borate, carbonate, citrate, acetate, phosphate and TRIS can be used to buffer the solutions of this invention to from about pH of 6.0 to about 8.0.

The ophthalmic compositions can also include one or more chelating components such as of ethylenediaminetetraacetic acid (EDTA) or the corresponding metal salts thereof such as the disodium salt. One possible alternative to the chelator EDTA or a possible combination with of ethylenediaminetetraacetic acid (EDTA) or the corresponding metal salts would be disodium disuccinate (EDDS) or a corresponding salt. Still another class of chelators includes alkylethylenediaminetriacetates such as lauroylethylenediaminetriacetate. See, U.S. Pat. No. 6,995,123 for a more complete description of such agents.

Tonicity agents may be utilized to adjust the osmolality of ophthalmic compositions here can be adjusted to hypotonic, isotonic or hypertonic relative to normal tears. These agents are usually simple salts such as sodium, potassium, calcium and magnesium chloride. These agents are usually simple salts such as sodium or potassium chloride. Low molecular weight organic components can also contribute to osmolality. Examples would be propylene glycol, glycerin, dextrose, mannitol and sorbitol. Preferably, the osmolality of the solutions of this invention are from about 150 to 450 mOsm/kg. More preferably, the osmolality of the solutions of this invention are from about 200 to 400 mOsm/kg. Most preferably, the osmolality of the solutions of this invention are in the range of 250 to 350 mOsm/kg.

Viscosifiers (demulcents) are often used to provide the ophthalmic compositions with a desired level of viscosity. Generally, ophthalmic solutions have a viscosity from about 1.0 cps to about 100.0 cps but more often are in the 10.0 cps to 30.0 cps range. There are many water soluble polymers to choose from, for example but not limited to, polyvinyl alcohol, polyvinyl pyrrolidone, hydroxyethyl cellulose, hydroxypropylmethyl cellulose, methyl cellulose, polyethylene oxide, polyethylene glycol, polyethyleneoxide, guar, carboxymethyl cellulose, carboxymethylhydroxyethyl cellulose, carboxyethylhydroxyethyl cellulose, carboxymethyl guar, carboxymethylhydroxypropyl guar, cellulose phosphate, chondoitin sulfate, N-carboxymethyl chitosan, alginates, sodium or potassium salts, xanthan, sodium or potassium salts, hyaluronic acid, sodium or potassium salt, glycomacropeptide, carboxyvinyl polymer, carbomer. Depending on the type of ophthalmic solution, whether an eye drop or a contact lens solution, the viscosity can vary widely. For the purposes of this invention preferably the viscosity can range from about 1.0 cps to about 500 cps. More preferably, the viscosity can range from about 1.0 cps to 400 cps. Most preferably, the viscosity can range from about 1.0 cps to 300 cps.

The compositions of this invention may include antimicrobial agents, disinfectants and preservatives in an effective amount. Antimicrobials known to the art include, but not limited to, alkyldimethyl benzylammonium chloride (BAK), chlorhexidene gluconate (CHG), polyhexamethylene biguanide (PHMB), Polyquaternium-1 (Polyquad®), parabens, other polyquats and sorbates, hydrogen peroxide, hydrogen peroxide/urea, sodium perborate, stabilized oxy-chloro complex (available commercially as OcuPure® from Advanced Medical Optics, Purite® from Allergan, and Purogene® from Biocide. The compositions of this invention may also include a co-preservative and/or chelating agent, such as, but not limited to, ethylenediaminetetraacetic acid (EDTA) and its salts.

The exemplary ophthalmic compositions described herein may also contain components that promote ocular health such as specific ions, such as Ca++, Zn++ and Mg++, Cu++, lutein, zeaxanthin and vitamins, such A, C and E.

The incorporation of a cooling agent in the disclosed ophthalmic compositions will provide relief of mild ocular irritation and enhance ocular comfort. Commercially, menthol has been incorporated into at least one product currently being marketed. Other cooling agents have been identified such as those disclosed in U.S. Patent Appl. 20100099771 which is hereby incorporated by reference in its entirety.

It may be desirable to include a radiation absorber in the ophthalmic compositions described herein. This may be particularly useful for individuals that suffer from VDT syndrome. Agents that help filter blue light are claimed to support the ocular health. Examples of agents that may be utilized to filter light are; lutein, zeaxanthin, vitamins such A and E, chlorophyllin and Xanthophyll.

Antioxidants are widely utilized in the food industry to protect certain ingredients from being oxidized. This is also true for ophthalmic compositions to protect certain components and provide longer stability and therefore product shelf life. While there are many antioxidant compounds available many are not suitable for food and cosmetic products. For that reason for the purposes of this invention a few examples of useful antioxidants are; ascorbic acid (vitamin C) and ethyl ascorbic acid, vitamin A, tocopherols, butylated hydroxyanisole (BHA), citric acid and citrates.

The ophthalmic compositions of this invention may be in the form of gels or ointments. Often times biopolymers derived from natural products are utilized to provide high viscosity gels and ointments. The types of galactomannans that may be used in the present invention are typically derived from guar gum, locust bean gum and tara gum. As used herein, the term “galactomannan” refers to polysaccharides derived from the above natural gums or similar natural or synthetic gums containing mannose or galactose moieties, or both groups, as the main structural components. Preferred galactomannans of the present invention are made up of linear chains of (1-4)-beta.-D-mannopyranosyl units with alpha.-D-galactopyranosyl units attached by (1-6) linkages. With the preferred galactomannans, the ratio of D-galactose to D-mannose varies, but generally will be from about 1:2 to 1:4. Galactomannans having a D-galactose:D-mannose ratio of about 1:2 is most preferred. Additionally, other chemically modified variations of the polysaccharides are also included in the “galactomannan” definition. For example, hydroxyethyl, hydroxypropyl and carboxymethylhydroxypropyl substitutions may be made to the galactomannans of the present invention. Non-ionic variations to the galactomannans, such as those containing alkoxy and alkyl (C1-C6) groups are particularly preferred when a soft gel is desired (e.g., hydroxylpropyl substitutions). Substitutions in the non-cis hydroxyl positions are most preferred. An example of non-ionic substitution of a galactomannan of the present invention is hydroxypropyl guar, with a molar substitution of about 0.4. Anionic substitutions may also be made to the galactomannans. Anionic substitution is particularly preferred when strongly responsive gels are desired.

The present invention compositions may comprise one or more galactomannan(s)). Preferably, the compositions will contain galactomannan and a borate compound as a gel promoter. The particular amounts of each will vary, depending on the particular gelling properties desired. Manipulating either the borate or galactomannan concentration provides stronger or weaker gelation at a given pH. If a strongly gelling composition is desired, then the borate or galactomannan concentration may be increased. If a weaker gelling composition is desired, such as a partially gelling composition, then the borate or galactomannan concentration may be reduced. Other factors may influence the gelling features of the compositions of the present invention, such as the nature and concentration of additional ingredients in the compositions, such as salts, preservatives, chelating agents and so on. Generally, preferred non-gelled compositions of the present invention, i.e., compositions not yet gel-activated by the eye, will have a viscosity of from about 5 00 to 1000 cps. Generally, preferred gelled compositions of the present invention, i.e., compositions gel-activated by the eye, will have a viscosity of from about 500 to 50,000 cps.

The galactomannans of the present invention may be obtained from numerous sources. Such sources include guar gum, locust bean gum and tara gum, as further described below. Additionally, the galactomannans may also be obtained by classical synthetic routes or may be obtained by chemical modification of naturally occurring galactomannans.

Guar gum is the ground endosperm of Cyamopisis tetragonolobus (L.) Taub. The water soluble fraction (85%) is called “guaran” (molecular weight of 220,000), which consists of linear chains of (1-4)-.beta.-D mannopyranosyl units with .alpha.-D-galactopyranosyl units attached by (1-6) linkages. The ratio of D-galactose to D-mannose in guaran is about 1:2. The gum has been cultivated in Asia for centuries and is primarily used in food and personal care products for its thickening property. It has five to eight times the thickening power of starch. Its derivatives, such as those containing hydroxypropyl or hydroxypropyltrimonium chloride substitutions, have been commercially available for over a decade. Guar gum may be obtained, for example, from Rhone-Polulene (Cranbury, N.J.), Hercules, Inc. (Wilmington, Del.) and TIC Gum, Inc. (Belcamp, Md.).

Locust bean gum or carob bean gum is the refined endosperm of the seed of the carob tree, ceratonia siliqua. The ratio of galactose to mannose for this type of gum is about 1:4. Cultivation of the carob tree is old and well known in the art. This type of gum is commercially available and may be obtained from TIC Gum, Inc. (Bekamp, Md.) and Rhone-Polulene (Cranbury, N.J.).

Tara gum is derived from the refined seed gum of the tara tree. The ratio of galactose to mannose is about 1:3. Tara gum is not produced in the United States commercially, but the gum may be obtained from various sources outside the United States.

In order to limit the extent of cross-linking to provide a softer gel characteristic, chemically modified galactomannans such as hydroxypropyl guar may be utilized. Modified galactomannans of various degree of substitution are commercially available from Rhone-Poulenc (Cranbury, N.J.). Hydroxypropyl guar with low molar substitution (e.g., less than 0.6) is particularly preferred.

Combination of the gelling system of the present invention with prior art gelling systems is also contemplated by the present invention. Such systems may include the inclusion of ionomers, such as xanthan, gellan, carageenan and carbomers, and thermogels, such as ethylhydroxyethyl cellulose.

In general, the compositions of the present invention can be used to administer various pharmaceutically active compounds to the eye. Such pharmaceuticals may include, but are not limited to, anti-hypertensive, anti-glaucoma, neuro-protective, anti-allergy, muco-secretagogue, angiostatic, anti-microbial, pain relieving and anti-inflammatory agents.

Examples of ophthalmic drugs include antibiotics such as tetracycline, chlortetracycline, bacitracin, neomycin, polymyxin, gramicidin, cephalexin, oxytetracycline, chloramphenicol, kanamycin, rifampicin, tobramycin, gentamicin, erythromycin and penicillin; antibacterials such as sulfonomides, sulfadiazine, sulfacetamide, sulfamethizole and sulfisoxazole, nitrofurazone and sodium propionate; antivirals including idoxuridine, trifluorothymidine, acyclovir, gancyclovir and interferon; non-antibiotic, anti-infection, anti-bacterial or anti-microbial drugs such as iodine based preparation triclosan, chlorhexidine, et al; anti-allergenics such as sodium cromoglycate, antazoline, methapyrine, chlorpheniramine, cetirizine and prophenpyridadine; anti-inflammatories such as hydrocortisone, hydrocortisoneacetate, dexamethasone, dexamethasone 21-phosphate, fluorocinolone, medrysone, prednisolone acetate, luoromethalone, hypothalamus releasing factor; beta adrenergic blockers such as timolol maleate, levobunclol HCl and betaxolol HCl; growth factors such as epidermal growth factor and fibronectin; carbonic anhydrase inhibitors such as dichlorphenamide, betamethasone, and triamcinolone and non-steroidal agents such as indomethacin, diclofenac, flurbiprofen, piroxicam, ibuprofen and acetylsalicylic acid; decongestants such as phenylephrine, naphazoline and tetrahydrozoline: miotics and anticholinesteras such as pilocarpine, acetylcholinechloride, physostigmine, eserine, carbachol, di-isopropylfluorophosphate, phospholineiodine, and demecarium bromide; mydriatics such as a tropine sulfate, cyclopentolate, homatropine, scopolamine, tropicamide, eucatropine, and hydroxyamphetamine; sympathomimetics such as epinephrine; immunological drugs such as vaccines and immunostimulants; hormonal agents such as estrogens, estradiol, progestational, progesterone, insulin, calcitonin, parathyroidhormone and peptide, vasopressin, acetazolamide and methazolamide and other drugs such as prostaglandins antiprostaglandins, and prostaglandin precursors; angiogenesis inhibitors such as liferative agents such as flurouracil and mitomycin.

Stevens-Johnson syndrome (SJS) is an acute inflammatory disease which often affects the skin and mucosal membranes including that of eyes. Many patients with SJS develop chronic ocular surface problems which may need immunosuppressive therapy. Among the drugs available to treat ocular inflammation are the glucocorticoids and drugs acting on immunophilins such as cyclosporine, tacrolimus and sirolimus. In solution form the compositions of this invention can be used to effectively deliver these drugs to the ocular surface. In a preferred embodiment the solutions of this invention are particularly useful as vehicles for the drug cyclosporine.

It is recognized that many biopolymers are sensitive to common sterilization procedures, e.g., heat sterilization. Heat sterilization can often lead to pronounced changes in the physico-chemical properties of the biopolymer such that the resulting sterile biopolymer is rendered unsuitable for its intended use.

Sterilization methods that are currently applied to medical materials include, for example, heat treatment, high-pressure vapor sterilization (e.g. autoclave sterilization), ethylene oxide gas (ETO) sterilization, supercritical carbon dioxide sterilization, radiation sterilization E-beam, and for solutions filtration through a 0.22 micron pore size filter

See for example, U.S. Pat. No. 6,891,035, U.S. Pat. No. 6,149,864, U.S. Pat. No. 5,621,093, U.S. Pat. No. 4,263,253, US 2006/0292030, U.S published application 20120295869 and U.S. published application 2007/0009578. Available sterilization methods are typically assessed in relation to the robustness of the particular biopolymer to be sterilized. For example, high-pressure vapor sterilization can be used for a biopolymer only to the extent that the biopolymer can endure high temperatures and high pressures. However, very few biopolymers including hyaluronic acid can endure such high temperatures and high pressures. EO sterilization could be useful because the process suppresses the thermal breakdown of the biopolymer. However, the presence of residual ethylene oxide in the composition can be problematic.

Ophthalmic compositions, such as solutions, are commonly autoclaved to affect sterility of the product. Another means of achieving a sterile solution is to use radiation such as ultra violet light or electron beam. The compositions of this invention can be processed by the above described methods.

The following detailed examples are illustrations of preferred embodiments. It should be clear that these are not intended to limit the scope of the present invention.

### EXAMPLES

### Example 1

The listed materials are utilized in the Examples that follow.

| | | |
|---|---|---|
| Polyquaternium-24 | Quatrisoft LM-200 | Amerchol |
| Polyquaternium-67 | SoftCAT polymer SL-100 | Amerchol |
| Polyquaternium-67 | SoftCAT polymer SX-1300X | Amerchol |
| Polyquaternium-67 | SoftCAT polymer SK-MH | Amerchol |
| Alginic acid Sodium salt | #180947 | Aldrich |
| Sodium CMC | 7HF PH | Hercules |
| Polycarbophil | Noveon AA1 | Lubrizol |
| Hyaluronic acid complex | MoistStar HA+* | Dow |
| Hyaluronic acid complex | Biopolymer HA-24** | Lipo chemical |
| Hyaluronic acid | Hylasome EG-10*** | Lipo chemical |
| Hyaluronic acid | Sodium hyaluronate**** | Lipo chemical |
| Hyaluronic acid | Grade HA-E2.0**** | Bloomage |
| | | Pharma |
| Hydroxyethyl cellulose | Natrosol 250M Pharm | Ashland |
| Glycomacropeptide | BioPure GMP | Davisco |

*MoistStar HA+ 3% active
**Biopolymer HA-24 #3% active
***Hylasome EG-10 0.18% active
****Hyaluronic acid >98% active

### Example 2

This example presents the properties of various cationic polymeric surfactants in aqueous solution both before and after autoclaving at 121° C. for 30 minutes (formulations in weight percent).

| INGREDIENT | A | B | C | D |
|---|---|---|---|---|
| Quatrisoft LM-200 | 0.2 | — | — | — |
| SoftCAT polymer SL-100 | — | 0.2 | — | — |
| SoftCAT polymer SX-1300X | — | — | 0.2 | — |
| SoftCAT polymer SK-MH | — | — | — | 0.2 |
| Purified water | q.s. | q.s. | q.s. | q.s. |
| | 100 | 100 | 100 | 100 |

### Physical Properties as Mixed

| | A | B | C | D |
|---|---|---|---|---|
| pH | 7.7 | 7.7 | 7.5 | 7.7 |
| Viscosity, cps | 2.1 | 27.8 | 37.0 | 31.7 |

Physical Properties after Autoclaving at 121° C. for 30 Minutes

| | A | B | C | D |
|---|---|---|---|---|
| pH | 7.3 | 8.2 | 8.1 | 8.2 |
| Viscosity, cps | 2.1 | 26.6 | 34.3 | 29.5 |

These results indicate that all the hydrophobic cationic celluloses tested are not significantly degraded by autoclaving. This is good evidence that these polymers can be processed in the conventional manner to produce sterile products.

### Example 3

The following example illustrates the preparation of an ophthalmic solution containing a hydrophobic cationic cellulose polymer utilizing a phosphate buffer system and PHMB as the preservative. The formulation presented here is prepared by weighing the Part A ingredients and then dissolving them in water with sufficient agitation to achieve complete dissolution. Part A was then autoclaved in a crimped top vial for 30 minutes at 121° C. to achieve sterilization. Part B is the preservative component PHMB and phosphate buffer in an aqueous solution and is not autoclaved.

| INGREDIENT | Part A |
|---|---|
| HEC 250M Pharm | 0.20 |
| SoftCat SX-1300X | 0.10 |
| Propylene glycol | 0.50 |
| Sodium chloride | 0.35 |
| EDTA | 0.10 |
| Purified water | 83.66 |

| INGREDIENT | Part B |
|---|---|
| PHMB | 5.0 ppm |
| Disodium phosphate | 0.28 |
| Potassium phosphate | 0.055 |
| Purified water | 14.76 |

In a laminar flow hood open the vial with Part A. Place Part B in a syringe equipped with a 0.22 micron filter and filter into Part A. Seal the vial and stir contents for 20 minutes to achieve a homogeneous solution. The physical properties of the completed solution are:

| Osmolality | 230 mOsm/kg |
|---|---|
| Viscosity | 16 cps |
| pH | 7.0 |

### Example 4

This example demonstrates the compatibility of three anionic polymers with Polyquaternium-24, a hydrophobic cationic cellulose, in aqueous solution. The following formulations, in weight percent, were prepared by mixing the ingredients for one hour at room temperature. After the solutions were thoroughly mixed certain attributes were assessed and presented below.

| INGREDIENT | AX | AY | AZ |
|---|---|---|---|
| Quatrisoft LM-200 | 0.1 | 0.1 | 0.1 |
| Sodium CMC | 0.2 | 0.1 | 0.05 |
| Purified water | q.s. | q.s. | q.s. |
| | 100 | 100 | 100 |
| CLARITY | VSH | VSH | VSH |
| LUBRICITY | L | L | L |
| SUBSTANTIVITY | High | High | High |
| | BX | BY | BZ |
| Quatrisoft LM-200 | 0.1 | 0.1 | 0.1 |
| Alginic acid Sodium salt | 0.2 | 0.1 | 0.05 |
| Purified water | q.s. | q.s. | q.s. |
| | 100 | 100 | 100 |
| CLARITY | VSH | VSH | VSH |
| LUBRICITY | L | L | L |
| SUBSTANTIVITY | High | High | High |
| | CX | CY | CZ |
| Quatrisoft LM-200 | 0.1 | 0.1 | 0.1 |
| Alginic acid Sodium salt | 0.2 | 0.1 | 0.05 |
| Purified water | q.s. | q.s. | q.s. |
| | 100 | 100 | 100 |
| CLARITY | H | H* | H* |
| LUBRICITY | L | L | L |
| SUBSTANTIVITY | Medium | Medium | Medium |

*Sight precipitate
KEY
VSH—Very slight Haze
H—Haze
L—Lubricious By rubbing between the fingers
SUBSTANTIVITY—Ability to coat glass and resist rinsing off with water

From the results it can be seen that the polymer complex formed was soluble with a slight haze due to hydrophobic interactions arising from the cationic polymeric surfactant. The formed complexes were found to be lubricious and had a strong affinity for glass surfaces.

### Example 5

This example presents the preparation of one of the complexes of this invention. The hydrophobic cationic cellulose is SoftCat SL-100 and the sodium hyaluronate is from Lipo Chemical. Three ratios were formulated 10/1, 1/1 and 1/10 weight percent of SoftCat to sodium hyaluronate. The formulations presented here are prepared by weighing the ingredients and then dissolving them in water with sufficient agitation to achieve complete dissolution.

| INGREDIENT | Complex A | Complex B | Complex C |
|---|---|---|---|
| SoftCat 100-SL | 0.20 | 0.10 | 0.02 |
| Sodium hyaluronate | 0.02 | 0.10 | 0.20 |
| Purified water | q.s | q.s. | q.s. |
| | 100 | 100 | 100 |
| Appearance | Clear | Clear | Clear |

These results indicate that the complexes of this invention can be successfully prepared over a wide weight ratio of hydrophobic cationic cellulose to sodium hyaluronate.

The above solutions were formulated to produce simple ophthalmic solutions. The formulations presented here are prepared by weighing the ingredients and then dissolving them in the complexes described above with sufficient agitation to achieve complete dissolution.

| INGREDIENT | A | B | C |
|---|---|---|---|
| Complex A | 50.0 | — | — |
| Complex B | — | 50.0 | — |
| Complex C | — | — | 50.0 |
| Sodium borate | 0.065 | 0.065 | 0.065 |
| Boric acid | 0.325 | 0.325 | 0.325 |
| Sodium chloride | 0.40 | 0.40 | 0.40 |
| Hydroxyethyl | 0.20 | 0.20 | 0.20 |
| cellulose 250M | | | |

The physical properties were determined on the above solutions.

| | | Viscosity, | Osmolality, |
|---|---|---|---|
| Solution | pH | cps | mOsm/kg |
| A | 7.3 | 285 | 350 |
| B | 7.3 | 267 | 360 |
| C | 7.3 | 250 | 360 |

### Example 6

The following example illustrates the preparation of an ophthalmic solution based on a complex of hydrophobic cationic cellulose polymer with Glycomacropeptide (GMP) which has been shown to be biologically active. GMP is an anionic peptide having an abundance of sialic acid groups. The formulations presented here are prepared by weighing the ingredients and then dissolving them in water with sufficient agitation to achieve complete dissolution. The solutions are then autoclaved in crimped top vials for 30 minutes at 121° C. to achieve sterilization.

| INGREDIENT | |
|---|---|
| HEC 250M Pharm | 0.40 |
| Quatrisoft LM-200 | 0.10 |
| BioPure GMP | 0.10 |
| Propyleneglycol | 0.50 |
| Disodium borate | 0.12 |
| Boric acid | 0.74 |
| EDTA | 0.05 |
| Purified water | q.s |
| | 100 |

The solution has a pH of 6.2 and a viscosity of 14.6 cps. One subject placed a drop of solution in the eye and reported that it was smooth with a comfortable feel.

### Example 7

The following example illustrates the preparation of ophthalmic solutions combining a hydrophobic cationic cellulose polymer with sodium hyaluronate to form a complex as described in this invention. The formulation presented here is prepared by weighing the ingredients and then dissolving them in water with sufficient agitation to achieve complete dissolution. The solution is then autoclaved in crimped top vials for 30 minutes at 121° C. to achieve sterilization.

| INGREDIENT | |
|---|---|
| HEC 250M Pharm | 0.30 |
| SoftCAT polymer SL-100 | 0.10 |
| Hylasome EG-10 | 0.10 |
| Propyleneglycol | 0.50 |
| Boric acid | 0.36 |
| Disodium borate | 0.06 |
| EDTA | 0.02 |
| Purified water | q.s |
| | 100 |

The sterilized solution was clear with no apparent undissolved material or precipitate. The physical properties are:

| pH | 7.3 | |
|---|---|---|
| Osmolality | 132 | mOsm/kg |
| Viscosity | 45.0 | cps |

One drop of the solution A was placed in the eye of two subjects and was judged to have a very smooth feel and was quite comfortable. The same was true of solution B. These solutions are non-preserved and represent an eye drop product for dry eye patients, contact lens wearers and computer users. Additionally these solutions are useful as a Rigid Gas Permeable lens insertion solution especially for large diameter lenses such as scleral and OrthoK.

### Example 8

The following example illustrates the preparation of an ophthalmic solution combining a complex of hydrophobic cationic cellulose polymer with sodium hyaluronate (Biopolymer HA-24) with a compound (trehalose) that has been shown to be useful in dry eye treatment. The formulations presented here are prepared by weighing the ingredients and then dissolving them in water with sufficient agitation to achieve complete dissolution. The solutions are then autoclaved in crimped top vials for 30 minutes at 121° C. to achieve sterilization.

| INGREDIENT | |
|---|---|
| HEC 250M Pharm | 0.40 |
| Biopolymer HA-24 | 9.50 |
| Propyleneglycol | 0.50 |
| Trehalose | 0.30 |
| Disodium borate | 0.007 |
| Purified water | q.s |
| | 100 |

The sterilized solutions were clear with no apparent undissolved material or precipitate. The physical properties are:

| | |
|---|---|
| pH | 7.0 |
| Viscosity | 21 cps |

The above sterilized solution was placed in a 40° C. incubator and samples were removed at various time points weeks and the properties were determined and compared to the initial properties.

| | Viscosity, cps | pH |
|---|---|---|
| Initial | 21 | 7.0 |
| Aged 10 weeks | 17 | 6.8 |
| Aged 18 weeks | 17 | 6.8 |

The solution was quite stable with very little change in properties over the test period.

### Example 9

The compositions of this invention can be in the form of a gel. The following composition is formulated from an aqueous Polyquaternium-24—sodium hyaluronate complex. The complex has a weight ratio of 1 to 1 Polyquaternium-24 to sodium hyaluronate. The total concentration of the complex is 0.18 weight percent in water. The product is a clear soft gel.

### Example 10

The following example explores the robustness of a hydrophobic cationic cellulose and a complex formed from hydrophobic cationic cellulose and sodium hyaluronate (MoistStar HA+) to sterilization by autoclaving. The formulations presented here are prepared by weighing the ingredients and then dissolving them in water with sufficient agitation to achieve complete dissolution. The solution is then placed in crimped top vials and autoclaved for 30 minutes at 121° C. to sterilize them.

| INGREDIENT | A | B |
|---|---|---|
| SoftCat polmer SL-100 | 0.10 | — |
| MoistStar HA+ | — | 6.67 |
| Purified water | q.s | q.s. |
| | 100 | 100 |
| Initial Viscosity cps | 14.7 | 21.5 |
| After autoclaving, viscosity cps | 12.0 | 13.2 |

It can be seen that autoclaving slightly reduced the viscosity of the hydrophobic cationic polymer solution. The viscosity of the hydrophobic cationic and sodium hyaluronate polymer complex solution exhibited a more noticeable change. It is not known whether this change was due to degradation or a rearranging of the complex structure in solution.

### Example 11

The following example illustrates the preparation of ophthalmic solutions combining a hydrophobic cationic cellulose polymer with sodium hyaluronate to form a complex as described in this invention. The formulations presented here are prepared by weighing the ingredients and then dissolving them in water with sufficient agitation to achieve complete dissolution. The solutions are then autoclaved in crimped top vials for 30 minutes at 121° C. to achieve sterilization.

| INGREDIENT | |
|---|---|
| HEC 250M Pharm | 0.20 |
| MoistStar HA+ | 6.67 |
| Glycerin | 0.50 |
| Propylene glycol | 0.50 |
| Sodium borate | 0.12 |
| Boric acid | 0.70 |
| Purified water | q.s. |
| | 100 |

Several crimped top bottles of solution were placed in a 40° C. incubator to age the samples. Periodically samples were pulled for physical property testing. The results of the aging study are presented below:

| | pH | Osmolality, mOsm/kg | Viscosity, cps |
|---|---|---|---|
| Before autoclaving | 7.1 | 252 | 27.5 |
| After autoclaving | 7.1 | 248 | 24.7 |
| Aged 3.5 months | 7.1 | 254 | 23.7 |
| Aged 5.5 months | 7.1 | 255 | 24.0 |

The results of the aging demonstrate that the solution of this example is very stable after 5.5 months in the 40° C. incubator.

### Example 12

The following example illustrates the preparation of an ophthalmic solution combining a complex of hydrophobic cationic cellulose polymer with sodium hyaluronate (MoistStar HA+) utilizing a phosphate buffer system. The formulation presented here is prepared by weighing the Part A ingredients and then dissolving them in water with sufficient agitation to achieve complete dissolution. Part A is then autoclaved in a crimped top vial for 30 minutes at 121° C. to achieve sterilization. Part B is the buffer system.

| INGREDIENT | Part A |
|---|---|
| HEC 250M Pharm | 0.20 |
| MoistStar HA+ | 6.67 |
| Glycerin | 0.50 |
| Propylene glycol | 0.50 |
| Purified water | 78.03 |
| | 85.90 |

| INGREDIENT | Part B |
|---|---|
| Disodium phosphate | 0.28 |
| Potassium phosphate | 0.055 |
| Purified water | 13.77 |
| | 14.11 |

In a laminar flow hood open the autoclaved crimped top vial with Part A. Place Part B in a syringe equipped with a 0.22 micron filter and filter into Part A. Seal the vial and stir contents for 20 minutes to achieve a homogeneous solution. The physical properties of the solution are:

Physical Properties

| Viscosity | 24.0 | cps |
|---|---|---|
| pH | 7.3 | |
| Osmolality | 183 | mOsm/kg |

### Example 13

The following example illustrates the preparation of an ophthalmic solution combining a complex of hydrophobic cationic cellulose polymer with sodium hyaluronate (MoistStar HA+) utilizing a borate buffer system and an oxychloro complex as a preservative. The formulation presented here is prepared by weighing the Part A ingredients and then dissolving them in water with sufficient agitation to achieve complete dissolution. The solutions are then autoclaved in crimped top vials for 30 minutes at 121° C. to achieve sterilization. Part B is the preservative component and is prepared from an oxychloro complex that is 2% active in water.

| INGREDIENT | Part A |
|---|---|
| HEC 250M Pharm | 0.20 |
| MoistStar HA+ | 6.67 |
| Glycerin | 0.50 |
| Propylene glycol | 0.50 |
| Sodium borate | 0.12 |
| Boric acid | 0.65 |
| Purified water | 86.53 |
| | 95.17 |

| INGREDIENT | Part B |
|---|---|
| Oxychloro complex, 2% | 0.28 |
| Purified water | 4.55 |
| | 4.83 |

In a laminar flow hood open the autoclaved vial with Part A. Place Part B in a syringe equipped with a 0.22 micron filter and filter into Part A. Seal the vial and stir contents for 20 minutes to achieve a homogeneous solution. The physical properties of the solution are:

| | | |
|---|---|---|
| Osmolality | 251 | mOsm/kg |
| Viscosity | 32.5 | cps |

| | |
|---|---|
| pH | 7.1 |

Stability of the solution is determined by placing samples in a 40° C. incubator for aging. After 7 weeks physical properties were determined.

| | | |
|---|---|---|
| Osmolality | 249 | mOsm/kg |
| Viscosity | 20 | cps |

| | |
|---|---|
| pH | 7.1 |

The solutions appear quite stable over the test period.

Rigid Gas Permeable (RGP) lenses require care solutions. One such system is the one bottle multi-purpose solution (MPS) that cleans and conditions the lens surface. The solution of this example was evaluated by two experienced RGP lens wearers as a MPS for a period of three months. It was reported that the lenses were very comfortable over the entire day of wear. Furthermore, it was also reported that the lenses were free of deposits indicating that the solution was effectively cleaning the lens surface.

### Example 14

The following example illustrates the preparation of ophthalmic solutions combining a hydrophobic cationic cellulose polymer with sodium hyaluronate to form a complex as described in this invention. The formulations presented here are prepared by weighing the ingredients and then dissolving them in water with sufficient agitation to achieve complete dissolution. The solutions are then autoclaved in crimped top vials for 30 minutes at 121° C. to achieve sterilization.

| INGREDIENT | |
|---|---|
| HEC 250M Pharm | 0.45 |
| MoistStar HA+ | 3.34 |
| Grade HA-E2.0 | 0.20 |
| Glycerin | 0.20 |
| Sodium chloride | 0.53 |
| Sodium borate | 0.12 |
| Boric acid | 0.64 |
| EDTA | 0.02 |
| Purified water | q.s. |
| | 100 |

After autoclaving the physical properties were determined and reported as follows:

| pH | 7.2 | |
|---|---|---|
| Viscosity | 297 | cps |
| Osmolality | 295 | mOsm/kg |

There is a new category of contact lens solution that is commonly referred to as an “insertion” solution. Currently an insertion solution is used with scleral lenses because they have large diameters and a large “bowl” into which the solution is placed. The instructions are—“Saline/application solution: When you are ready to apply your lens, remove it from the conditioning solution. Rinse with saline. Fill the bowl of the lens with non-preserved saline (or other non-preserved solution as recommended by your provider), and apply to the surface of your eye.” Saline has essentially no viscosity and tends to run off the lens during insertion and when the lens is placed on the eye air bubbles are trapped between the lens and the eye severely compromising vision. This also can occur with other large diameter RGP lenses including Ortho K lenses. The solution presented here was formulated as an ideal example of a contact lens insertion solution.

Two experienced RGP lens wearers evaluated the solution in this Example as a lens insertion solution. After several days of testing both wearers reported that the solution was very comfortable upon insertion and their lenses remained comfortable throughout the day.

The various methods and techniques described above provide a number of ways to carry out the invention. Of course, it is to be understood that not necessarily all objectives or advantages described may be achieved in accordance with any particular embodiment described herein. Thus, for example, those skilled in the art will recognize that the methods may be performed in a manner that achieves or optimizes one advantage or group of advantages as taught herein without necessarily achieving other objectives or advantages as may be taught or suggested herein.

Furthermore, the skilled artisan will recognize the interchangeability of various features from different embodiments. Similarly, the various features and steps discussed above, as well as other known equivalents for each such feature or step, can be mixed and matched by one of ordinary skill in this art to perform methods in accordance with principles described herein. Although the invention has been disclosed in the context of certain embodiments and examples, it will be understood by those skilled in the art that the invention extends beyond the specifically disclosed embodiments to other alternative embodiments and/or uses and obvious modifications and equivalents thereof. Accordingly, the invention is not intended to be limited by the specific disclosures of preferred embodiments herein, but instead by reference to claims attached hereto.

1. An ophthalmic composition comprising:
a cationic polymeric surfactant derived from a polysaccharide, wherein the cationic polymeric surfactant is represented by the overall structural formula: 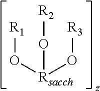
wherein: 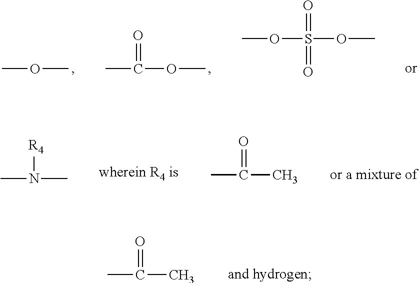
R_{sacch} is the residue of a polysaccharide repeat unit;
z is from 50 to about 20,000; and
each R₁, R₂ and R₃ is individually represented by the substituent structural formula: 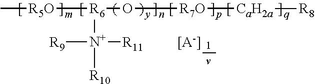
wherein:
A is an anion;
a is an integer of from 1 to about 3;
m is an integer of from 0 to about 6;
n is an integer of from 0 to about 3, provided that the level of cationic substitution, CS, defined by the average moles of quaternary nitrogen atoms per mole polysaccharide repeat unit is greater than 0;
p is an integer of from 0 to about 6;
q is 0 or 1;
each R₅ and R₇ is individually ethylene, a propylene or a hydroxypropylene;
R₆ is a di- or trivalent, branched or straight chain, saturated or unsaturated hydrocarbon having from 2 to about 4 carbon atoms, provided there are at least 2 carbon atoms between the nitrogen atom and any oxygen atom;
R₈ is hydrogen, hydroxyl, Rₕ, carboxyl or alkali metal or amine carboxylate, provided that when q is 0 then R₈ is hydrogen or Rₕ;
each R₉, R₁₀ and R₁₁ is individually Rₕ, alkyl, aryl, aralkyl, alkaryl, cycloalkyl, alkoxyaryl or alkoxyalkyl, having at least two carbon atoms separating the oxygen atom in the alkoxyaryl or alkoxyalkyl group from the nitrogen atom;
Rₕ is a hydrophobic group containing an alkyl group having at least 8 carbon atoms;
v is equal to the valence of A;
y is 0 or 1, provided that when y is 0 then p and q are 0 and R₈ is hydrogen;
with the proviso that at least one Rₕ group is present such that the extent of hydrophobic group substitution, HS, defined by the average moles of said hydrophobic groups per mole of polysaccharide repeat unit, is greater than 0;
a demulcent; and
a buffering agent.

2. The ophthalmic composition of claim 1, wherein the composition is in the form of a solution, gel, ointment or dispersion that is suitable for instilling into an eye.

3. The ophthalmic composition of claim 1, wherein the cationic polymeric surfactant is present in the amount of from about 0.01 to about 3.0 weight percent.

4. The ophthalmic composition of claim 1, wherein the cationic polymeric surfactant is derived from cellulose.

5. (canceled)

6. The ophthalmic composition of claim 1, further comprising at least one of tonicity adjusting agents, preservatives and disinfectants.

7. The ophthalmic composition of claim 1, wherein the cationic polymeric surfactant is Polyquaternium-24 (Cas Number 98616-25-2) or Polyquaternium-67.

8. The ophthalmic composition of claim 1, further comprising an anionic polymer comprising one of sodium hyaluronate and a potassium hyaluronate, wherein the cationic polymeric surfactant and the anionic polymer form a complex.

9. The ophthalmic composition of claim 8, wherein the cationic polymeric surfactant is present in the amount of from about 0.01 to about 3.0 weight percent and the sodium hyaluronate or potassium hyaluronate is present in the amount of from about 0.01 to about 5.0 percent by weight.

10. The ophthalmic composition of claim 1, wherein the composition comprises and aqueous solution and the cationic polymeric surfactant comprises a hydrophobic cationic cellulose.

11. The ophthalmic composition of claim 1, further comprising an ophthalmic drug.

12. The ophthalmic composition of claim 8, wherein the cationic polymeric surfactant comprises a hydrophobic cationic cellulose and the anionic polymer comprises sodium hyaluronate and a ratio between the weight percent of the hydrophobic cationic cellulose compared to the sodium hyaluronate is between 1/10 and 10/1.

13. The ophthalmic composition of claim 12, wherein the buffering agent comprises a mixture of sodium borate and boric acid, and the demulcent comprises hydroxyethyl cellulose and the composition further comprises a tonicity agent in the form of sodium chloride.

14. The ophthalmic composition of claim 8, wherein the cationic polymeric surfactant comprises a hydrophobic cationic cellulose and the anionic polymer comprises sodium hyaluronate and the buffering agent comprises a mixture of sodium borate and boric acid and the composition further comprises glycerin, and a preservative component comprising an oxychloro complex.

15. A method for treating at least one condition selected from the group consisting of dry eye syndrome, keratitis sicca, xerophthalmia, keratoconjunctivitis sicca, ocular discomfort, rhinological allergic complications, Sjogren's syndrome, chronic low blink rate (VDT syndrome), recovery from LASIK vision correction surgery, and Stevens Johnson syndrome comprising the step of administering a solution that contains the composition of claim 1.

16. The method of claim 15, wherein the cationic polymeric surfactant is present in the amount of from about 0.01 to about 3.0 weight percent.

17. The method of claim 15, further comprising at least one of preservatives and disinfectants.

18. The method of claim 15, wherein the cationic polymeric surfactant is Polyquaternium-24 (Cas Number 98616-25-2) or Polyquaternium-67.

19. The method of claim 15, wherein the composition further includes an anionic polymer that complexes with the cationic polymeric surfactant, the anionic polymer comprising one of sodium hyaluronate and potassium hyaluronate.

20. The method of claim 19, wherein the cationic polymeric surfactant is present in the amount of from about 0.01 to about 3.0 weight percent and the sodium hyaluronate or potassium hyaluronate is present in the amount of from about 0.01 to about 5.0 percent by weight.

21. The method of claim 15, wherein the composition comprises an aqueous solution and the cationic polymeric surfactant comprises a hydrophobic cationic cellulose.

22. The method of claim 15, further comprising an ophthalmic drug.

23. The method of claim 19, wherein the cationic polymeric surfactant comprises a hydrophobic cationic cellulose and the anionic polymer comprises sodium hyaluronate and a ratio between the weight percent of the hydrophobic cationic cellulose compared to the sodium hyaluronate is between 1/10 and 10/1.

24. (canceled)

25. The ophthalmic composition of claim 1, wherein the demulcent is selected from the group consisting of: polyvinyl alcohol, polyvinyl pyrrolidone, hydroxyethyl cellulose, hydroxypropylmethyl cellulose, methyl cellulose, polyethylene oxide, polyethylene glycol, polyethyleneoxide, guar, carboxymethyl cellulose, carboxymethylhydroxyethyl cellulose, carboxyethylhydroxyethyl cellulose, carboxymethyl guar, carboxymethylhydroxypropyl guar, cellulose phosphate, chondoitin sulfate, N-carboxymethyl chitosan, alginates, sodium or potassium salts, xanthan, hyaluronic acid, glycomacropeptide, carboxy vinyl polymer, and carbomer.

26. The ophthalmic composition of claim 1, wherein the buffering agent comprises a mixture of sodium borate and boric acid.

27. A method for forming an ophthalmic composition comprising the steps of:
adding a cationic polymeric surfactant to an aqueous solution including a demulcent and a buffering agent to form the ophthalmic composition, wherein the cationic polymeric surfactant being derived from a polysaccharide, wherein the cationic polymeric surfactant is represented by the overall structural formula: 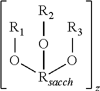
wherein: 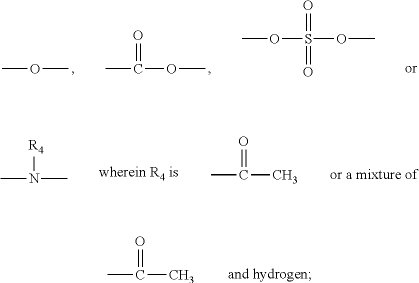
R_{sacch} is the residue of a polysaccharide repeat unit;
z is from 50 to about 20,000; and
each R₁, R₂ and R₃ is individually represented by the substituent structural formula: 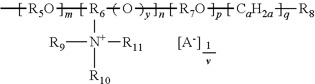
wherein:
A is an anion;
a is an integer of from 1 to about 3;
m is an integer of from 0 to about 6;
n is an integer of from 0 to about 3, provided that the level of cationic substitution, CS, defined by the average moles of quaternary nitrogen atoms per mole polysaccharide repeat unit is greater than 0;
p is an integer of from 0 to about 6;
q is 0 or 1;
each R₅ and R₇ is individually ethylene, a propylene or a hydroxypropylene;
R₆ is a di- or trivalent, branched or straight chain, saturated or unsaturated hydrocarbon having from 2 to about 4 carbon atoms, provided there are at least 2 carbon atoms between the nitrogen atom and any oxygen atom;
R₈ is hydrogen, hydroxyl, Rₕ, carboxyl or alkali metal or amine carboxylate, provided that when q is 0 then R₈ is hydrogen or Rₕ;
each R₉, R₁₀ and R₁₁ is individually Rₕ, alkyl, aryl, aralkyl, alkaryl, cycloalkyl, alkoxyaryl or alkoxyalkyl, having at least two carbon atoms separating the oxygen atom in the alkoxyaryl or alkoxyalkyl group from the nitrogen atom;
Rₕ is a hydrophobic group containing an alkyl group having at least 8 carbon atoms;
v is equal to the valence of A;
y is 0 or 1, provided that when y is 0 then p and q are 0 and R₈ is hydrogen;
with the proviso that at least one Rₕ group is present such that the extent of hydrophobic group substitution, HS, defined by the average moles of said hydrophobic groups per mole of polysaccharide repeat unit, is greater than 0.

28. The method of claim 27, wherein the demulcent is selected from the group consisting of: polyvinyl alcohol, polyvinyl pyrrolidone, hydroxyethyl cellulose, hydroxypropylmethyl cellulose, methyl cellulose, polyethylene oxide, polyethylene glycol, polyethyleneoxide, guar, carboxymethyl cellulose, carboxymethylhydroxyethyl cellulose, carboxyethylhydroxyethyl cellulose, carboxymethyl guar, carboxymethylhydroxypropyl guar, cellulose phosphate, chondoitin sulfate, N-carboxymethyl chitosan, alginates, sodium or potassium salts, xanthan, hyaluronic acid, glycomacropeptide, carboxy vinyl polymer, and carbomer.

29. The method of claim 27, wherein the buffering agent comprises a mixture of sodium borate and boric acid.
